(19) United States
(12) Patent Application Publication
(10) Pub. No.: US 2024/0342349 A1
(43) Pub. Date: Oct. 17, 2024

(54) BREAST CONTACTING ELEMENT

(71) Applicant: MAYBORN (UK) LIMITED, Tyne and Wear (GB)

(72) Inventors: Nicholas CUDWORTH, Tyne and Wear (GB); Joshua HUME, Tyne and Wear (GB)

(21) Appl. No.: 18/294,822

(22) PCT Filed: Aug. 3, 2022
(86) PCT No.: PCT/GB2022/052040

§ 371 Date: Feb. 2, 2024

(30) Foreign Application Priority Data
Aug. 5, 2021 (GB) 2111318.8

(51) Int. Cl. A61M 1/06

(52) U.S. Cl. CPC A61M 1/0697; A61M 1/066; A61M 2205/0216; A61M 2207/10

(57) ABSTRACT

The present invention relates to a breast contacting element for a breast pump assembly and a method of forming the same. More specifically, there is provided a breast contacting element for a breast pump assembly, including an opening adapted to receive a nipple of a user's breast: a breast contacting surface extending from the opening and comprising a deformable portion for engaging the user's breast; and a channel configured for conveying a fluid pressure from the opening to the deformable portion such that the deformable portion deflects in response to the fluid pressure applied thereto. Specifically, the method includes forming a support element of a first material, the support element comprising an orifice and a channel extending from the orifice, and injecting a second material onto the support element so as to form a deformable portion across the orifice.

The present invention relates to a breast contacting element for a breast pump assembly and a method of forming the same.

### INTRODUCTION

Breast pumps are used by nursing mothers to allow them to conveniently express their breast milk for storage and later feeding to infants. Such breast pumps stimulate milk expression by mimicking the action of a baby by applying suction to the breast. Negative pressure applied to the breast in short or cyclical pulses provides efficient milk expression.

In use a breast contacting element of a breast pump assembly is positioned onto the breast so that at least the nipple is received within a narrowed opening or recess within the breast contacting element. Once positioned, a negative pressure source is activated to apply suction to the breast via the narrowed opening or recess, thereby encouraging expression of breast milk.

Breast contacting elements for a breast pump assembly are known in which a massaging portion is provided as a flexible liner mounted to a rigid supporting member, for example a horn member. In certain configurations, openings in the rigid supporting member allow a user to apply manual pressure to a massaging portion in an attempt to encourage expression. Alternatively, the flexible liner may be configured to respond to the negative pressure from the narrowed opening or recess to deform and engage with the breast.

A problem with known breast contacting elements is that they can be awkward to dissemble and assemble in breast pump assemblies, as is required for cleaning and sterilising breast pump assemblies. Coupling a flexible portion to a supporting member can result in unreliable sealing between the parts, reducing the efficiency of the negative pressure. Such arrangements require additional cleaning and assembly time for the user, and require manufacture of multiple parts, increasing costs. Breast contacting elements which rely on manual manipulation necessarily require the user to repeatedly squeeze the massaging portion with their hand to try to massage the breast. As the user must already hold the breast pump in place against the breast with one hand then manual massaging the breast necessarily becomes a two-handed activity.

Manual manipulation also requires physical effort by the user to compress the massaging portion against the breast, meaning that as the user's hand tires then effectiveness is reduced over time. Furthermore, devices which rely on the user for massaging are limited to applying pressure to small areas of the breast using an opposing thumb and finger action. This limits the effectiveness of the action because only a small proportion of the breast can be massaged at any time.

A further known problem of breast contacting elements is that the response of a flexible region to a negative pressure cannot be reliably controlled. A flexible region will flex where its surface is exposed to negative pressure from the negative pressure source.

Thus, when a breast pump assembly is positioned onto the breast, the breast conforms to the breast contacting element such that only a small portion of the flexible region adjacent the nipple remains uncovered. The remainder of the breast contacting surface is engaged by the breast. Consequently, only the uncovered flexible region of the breast contacting element, typically the region adjacent the nipple, is able to respond to negative pressure. Thus, the breast contacting element provides inadequate massaging where it engages the breast.

Accordingly, it would be useful to provide a breast contacting element and a method of forming a breast contacting element which provides an efficient massaging effect to a breast received within a breast pump assembly. In particular, it would be useful for a breast contacting element to be capable of efficiently massaging a breast in regions where the breast is in engaged with the breast contacting element.

It would also be useful to provide a breast contacting element as a single part, which is convenient and easy to use with a breast pump assembly. In particular, it would be useful to provide a breast contacting element that provides effective massaging within a breast pump assembly and which permits single-handed use for the user.

### SUMMARY OF THE INVENTION

According to a first aspect of the invention, there is provided a breast contacting element for a breast pump assembly, including:
an opening adapted to receive a nipple of a user's breast;
a breast contacting surface extending from the opening and comprising a deformable portion for engaging the user's breast; and
a channel configured for conveying a fluid pressure from the opening to the deformable portion such that the deformable portion deflects in response to the fluid pressure applied thereto.

Aptly, the deformable portion may be domed.

Aptly, the deformable portion may be configured to invert or flex in response to the fluid pressure applied to the deformable portion

Aptly, the deformable portion may have a thickness in the range of from 0.5 mm to 2 mm, preferably in the range of from 1 mm to 1.5 mm.

Aptly, the deformable portion may include an elastomer having a material hardness in the range of from Shore A 20 to Shore A 80, and preferably in the range of from Shore A 25 to Shore A 50.

Aptly, the breast contacting surface may include a plurality of deformable portions.

Aptly, a plurality of deformable portions may include at least 60% of the breast contacting surface, and preferably include in the range of from 70% to 80% of the breast contacting surface. That is, the deformable portions of the plurality of deformable portions form at least 60% of the surface area of the breast contacting surface, and preferably form from 70% to 80% of the surface area of the breast contacting surface.

Aptly, the breast contacting surface flares outwardly from the opening.

Aptly, the breast contacting surface flares outward from the opening at an increasing angle relative to a central axis of the opening. The breast contacting surface includes a first flared surface extending proximal to the opening and a second flared surface extending distal to the opening. The first flared surface extends at a first angle relative to the central axis of the opening and the second flared surface extends at a second angle relative to the central axis, wherein the second angle is greater than the first angle. In certain examples, the first angle is in a range of from greater than 0° to 30°, preferably from 10° to 20°. In certain examples, the second angle is in a range from 20° to 70°, preferably in the range from 30° to 60°. In certain examples, the second angle is at least 20° larger, preferably at least 30° larger, and more preferably at least 40° larger, than the first angle.

Aptly, the channel for conveying a fluid pressure from the opening to the deformable portion extends to the second flared surface.

Aptly, the deformable portion extends from the first flared surface to the second flared surface.

Aptly, the breast contacting element further may include a support element including an orifice, and the deformable portion may be formed across the orifice.

Aptly, the orifice may extend though the support element.

Aptly, the deformable portion may be coupled to an edge of the orifice.

Aptly, the channel may be formed in the breast contacting surface.

Aptly, the channel may include a recess extending along the breast contacting surface.

Aptly, the breast contacting element may include a distal opening for operably connecting the breast contacting element to a fluid pressure source, and the channel may extend towards the distal opening.

Aptly, the breast contacting element may include a conduit extending from the opening to the distal opening.

Aptly, the channel may extend along an inner surface of the conduit.

Aptly, the breast contacting surface may include a textured region.

Aptly, the textured region may be provided on the deformable portion.

According to a second aspect of the invention, there is provided a breast pump assembly including:
a conduit, operably connectable to a fluid pressure source, the conduit having an opening adapted to receive a nipple of a user's breast,
a breast contacting surface extending from the opening and comprising a deformable portion for engaging the user's breast; and
a channel configured for conveying a fluid pressure generated by the fluid pressure source from the opening to the deformable portion such that the deformable portion deflects in response to the fluid pressure applied thereto.

According to a third aspect of the invention, there is provided a method of forming a breast contacting element for a breast pump assembly, the method including:
forming a support element of a first material, the support element comprising an orifice and a channel extending from the orifice, injecting a second material onto the support element so as to form a deformable portion across the orifice.

Aptly, the breast contacting element may include a breast contacting surface, and the deformable portion may form at least a portion of the breast contacting surface.

Aptly, the orifice may be a plurality of orifices and the step of forming the support element may include forming an inlet manifold for receiving the injected second material.

Aptly, the inlet manifold may be configured such that during the step of injecting the second material onto the support element the inlet manifold directs the injected second material to each of the plurality of orifices.

Aptly, the breast contacting element may include a distal opening for coupling the breast contacting element to a breast pump, and the inlet manifold may be configured to further direct the injected second material to form a sealing element at the distal opening.

Certain examples provide a more reliable response to fluid pressure due to the improved combination of a dimensionally-stable support with effective deformable portions.

Certain examples provide a more effective massaging effect when expressing breast milk. In particular, a breast contacting element may be provided which enables a deformable portion to deflect even when engaged with the breast. That is, the deformable portion deflects even in regions of the breast contacting surface which are in contact with the breast.

Certain examples provide an improved massaging action. That as, in certain examples, massaging pressure is provided to an increased area of the breast. Additionally, an improved stripping action, akin to a natural feeding action, may be provided.

Certain examples provide more efficient milk expression. Thus, milk may be expressed at an improved rate, shortening expressing time for a user. Furthermore, a breast contacting element may be used with reduced fluid pressure, thereby increasing comfort for the user.

Certain examples provide a breast contacting element that is simple to assemble and disassemble within a breast pump assembly. Furthermore, the breast pump assembly may be conveniently operated single-handed by a user.

Certain examples provide a method of forming a breast contacting element, including effective deformable portions, which is simpler and cheaper to manufacture.

It will be appreciated that any of the features described above in relation to the first aspect of the invention may apply equally to the second or third aspects of the invention. That is, any features described above under the first aspect may be combined with the breast pump assembly of the second aspect or with the method of the third aspect. Also, any features described above under the second aspect may be combined with the method of the third aspect.

### BRIEF DESCRIPTION OF THE DRAWINGS

Embodiments of the invention are now described, by way of example only, with reference to the accompanying drawings, in which:

FIG. 1 shows a perspective view of a breast contacting element;

FIG. 2 shows (a) a side view and (b) a cross-sectional view of a further breast contacting element;

FIG. 3 shows (a) a top view and (b) a bottom view of the breast contacting element of FIG. 2;

FIG. 4 shows a cross-sectional view of the breast contacting element of FIG. 2 engaged with a breast;

FIG. 5 shows a series of cross-sectional views (a) to (d) of the arrangement of FIG. 4 in use;

FIG. 6 shows a method of forming a breast contact element for a breast pump assembly;

FIG. 7 shows (a) an upper and (b) a lower perspective view of a support element formed in the first step of the method of FIG. 6;

FIG. 8 shows a perspective view of a breast contacting element formed in the second step of the method of FIG. 6;

FIG. 9 shows perspective views of a breast contacting element responding to a fluid pressure;

FIG. 10 shows a perspective view of a further breast contacting element;

FIG. 11 shows a perspective view of a yet further breast contacting element; and

FIG. 12 shows a perspective view of another breast contacting element.

In the drawings, like reference numerals refer to like parts.

### DETAILED DESCRIPTION

Certain terminology is used in the following description for convenience only and is not limiting. The words ‘lower’ and ‘upper’ designate directions in the drawings to which reference is made and are with respect to the described component when assembled and mounted. The words ‘inner’, ‘inwardly’ and ‘outer’, ‘outwardly’ refer to directions toward and away from, respectively, a designated centreline or a geometric centre of an element being described (e.g. central axis), the particular meaning being readily apparent from the context of the description.

Further, unless otherwise specified, the use of ordinal adjectives, such as, ‘first’, ‘second’, ‘third’ etc. merely indicate that different instances of like objects are being referred to and are not intended to imply that the objects so described must be in a given sequence, either temporally, spatially, in ranking or in any other manner.

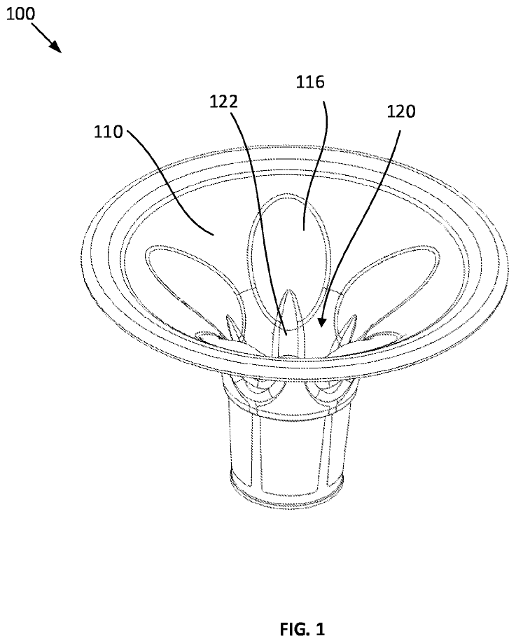

Referring now to FIG. 1, there is shown a breast contacting element 100 of a breast pump assembly which includes an opening 120 adapted to receive a nipple of a user. That is, the opening 120 of the breast contacting element 100 is sized and shaped such that a user may position their nipple therethrough so that the nipple is comfortably received for milk expression.

The breast contacting element 100 includes a breast contacting surface 110 extending from the opening 120. The breast contacting surface 110 includes a deformable portion 116 for engaging the user's breast. That is, the deformable portion 116 engages the user's breast as the nipple is received into the opening 120.

The breast contacting element 100 further includes a channel 122. The channel 122 is configured for conveying a fluid pressure from the opening 120 to the deformable portion 116, such that the deformable portion 116 deflects in response to the fluid pressure applied thereto. In this way, the deformable portion 116 which engages the breast is able to deflect in response to fluid pressure, such as negative pressure used during expressing, to provide a massaging effect to the breast.

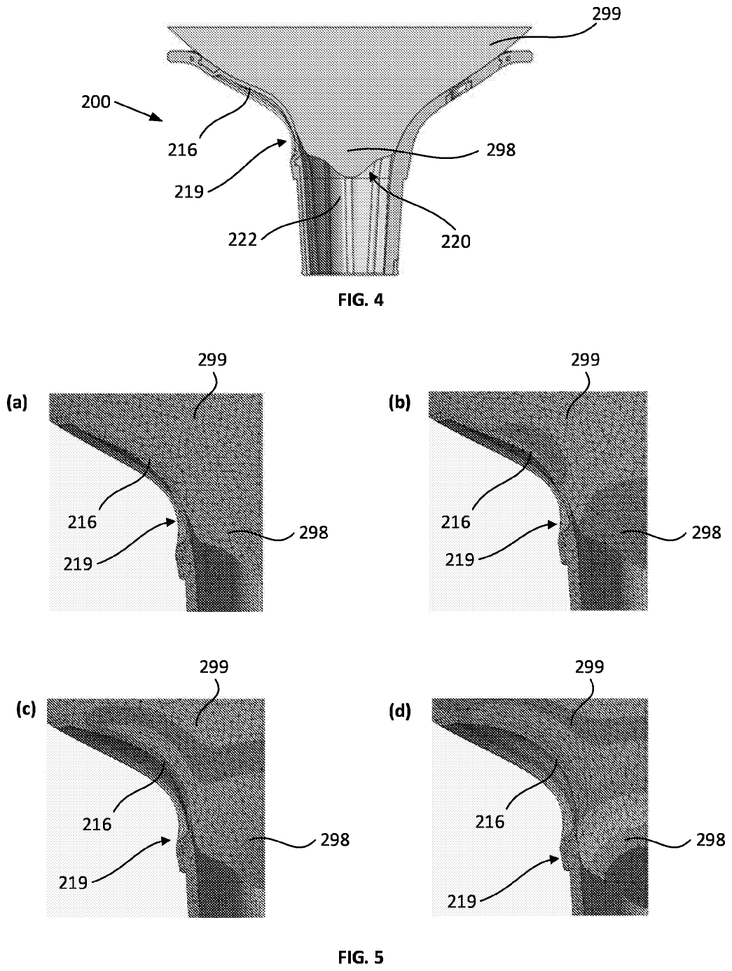

Furthermore, the deformable portion 116 remains responsive even when the breast contacting element 100 engages with a breast. By providing a channel 122, the breast contact element 100 can be used to convey a fluid pressure to the deformable portion 116 even as the deformable portion 116 is in contact with the breast. The channel 122 conveys fluid pressure beyond an innermost, or centremost, engagement limit of the breast contacting surface 100 with the breast, such as the engagement limit 219 described below in reference to FIGS. 4 and 5 (a) to (d). Consequently, the deformable portion 116 will deflect when a fluid pressure is provided from the opening 120 even where the deformable portion 116, or a portion thereof, is located beyond the engagement limit.

The deformable portion 116 may be domed. That is the deformable portion 116 may be provided in a first position to bulge away from, or be generally convex to, the breast contacting surface 110 so as to project in a direction towards the breast when engaged. Alternatively, the deformable portion 116 may be provided in a first position generally concave to the breast contacting surface 110 so as to be, recessed in a direction away the breast when engaged.

The deformable portion 116 may be configured to invert or flex in response to the fluid pressure applied to the deformable portion 116. That is, the deformable portion 116 may respond to a fluid pressure to move from a first position towards a second position, in which the deformable portion 116 has moved relative to the breast. In this way, the deformable portion 116 may generate a substantial movement relative to the breast so as to provide a pronounced massaging effect.

The breast contacting surface 110 engages the user's breast as the nipple is received into the opening 120. The breast contacting element 100 may thereby sealingly engage the user's breast such that, during use, fluid pressure from the opening 120 can be maintained within the opening 120 and within the channel 122 without ambient air leaking in. The sealing engagement also prevents expressed milk leaking from the breast contacting element 100.

To use the breast contacting element 100 to express milk in a breast pump assembly the breast contacting element 100 is fluidly connected to a negative pressure source to enable suction to be applied to a user's breast from the opening 120. The breast pump assembly may thus include the negative pressure source, which may be a manual breast pump, in which the negative pressure source is actuated by a user so that suction is generated by a manual action of a user. For example, a user may actuate a mechanical pump or manually deform a resilient container.

Alternatively, the breast pump assembly may include an electric breast pump, in which the negative pressure source is a suitable electrical apparatus, such as an electromechanical pump. The breast pump assembly may be provided as a wearable breast pump assembly, that is provided as an assembly which may be worn by the user so as to be positioned on their breast and operated without the user needing to hold the assembly.

With the breast contacting element 100 engaged with user's breast, the user activates the negative pressure source to provide a negative pressure within the opening 120. The negative pressure is conveyed from the opening 120 along the channel 122 to the deformable portion 116, causing the deformable portion 116 to respond to the negative pressure. The deformable portion 116 responds by deflecting so as to move relative to the breast, thereby providing a massaging effect. The channel 122 ensures that the deformable portion 116 is exposed to the negative pressure even when covered by the breast, that is even when engaged with the breast such that a substantial portion of the deformable portion 116 is in contact with the breast. Consequently, the deformable portion 116 provides a reliable response to negative pressure whenever the deformable portion 116 is engaged with the breast.

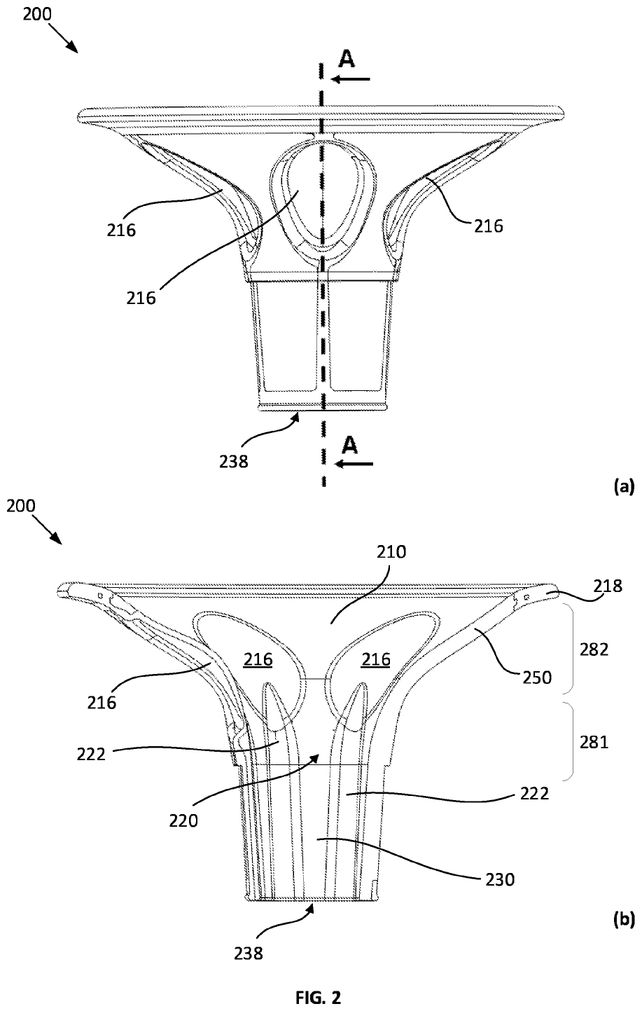

Referring now to FIGS. 2 (a) and 2 (b), there are shown side and cross-sectional views of a breast contacting element 200 of a breast pump assembly. The cross-section view of FIG. 2 (b) is taken in a plane extending through the central longitudinal axis A-A of the breast contacting element, as shown on FIG. 2 (a). Where features are the same as a previous example, the reference numbers are the same, other than the initial digit is a “2”. The breast contacting element 200 is substantially the same as breast contacting element 100 of FIG. 1 and includes a plurality of deformable portions 216 on the breast contacting surface 210. That is, the breast contacting surface 210 extends from the opening 220 and is provided with a plurality of deformable portions 216 arranged around the opening 220.

Each deformable portion 216 of the plurality of deformable portions 216 is provided with a channel 222 configured to convey fluid pressure from the opening 220. In this way, fluid pressure within the opening 220 provides a responsive deflection in each deformable portion 216. Consequently, the plurality of deformable portions 216 enable a massaging effect to be provided in a plurality of directions from the breast contacting surface 210, thereby increasing effectiveness.

In alternative configurations any number of channels and deformable portions may be provided in any suitable configuration so as to convey fluid pressure from an opening. For example, a channel may convey fluid pressure to one or more deformable portions. Or, one or more channels may convey fluid pressure to each deformable portion.

Each channel 222 is formed in the breast contacting surface 210. In this way, the channel 222 forms an elongated cavity in the breast contacting surface 210. The channel 222 may be provided as a recess extending along the breast contacting surface 210. That is, the channel 222 is formed as an open channel. The open channel includes a profile shape such that, with a breast engaged against the breast contacting surface 210, at least a portion of the profile shape remains unblocked along the channel.

In alternative arrangements, the channel may be formed as a closed channel in the breast contacting surface. For example, the channel may be provided as a duct or lumen on or within the breast contacting surface, and which extends from the opening to a deformable portion.

In this example, the deformable portions 216 are arranged so as to be spaced apart around the breast contacting surface 210. In this way, the breast contacting surface 210 includes a plurality of deformable portions 216 separated by regions of a support element 250.

Referring in particular to FIG. 2 (b) a cross-sectional view of a representative deformable portion 216 is shown. The deformable portion 216 is provided so as to provide sufficient flexibility to deflect in response to fluid pressures suitable for expressing milk. Preferably, the deformable portion 216 is formed from an elastomer material, for example a thermoplastic elastomer or a silicone rubber. Consequently, the response to fluid pressure by the deformable portion 216 may be adjusted by modifying the deformable portion using, for example, at least one of appropriate selection of material, material hardness, and material thickness so as to provide a deflection which generates effective massaging of the breast.

For certain examples, a thickness of the deformable portion 216 is in the range of from 0.5 mm to 2 mm. Preferably, the thickness of the deformable portion 216 is in the range of from 1 mm to 1.5 mm.

For certain examples, a deformable portion is formed of an elastomer having a material hardness in the range of from Shore A 20 to Shore A 80. Preferably, the elastomer has a material hardness in the range of from Shore A 25 to Shore A 50.

For certain examples, the deformable portions of the plurality of deformable portions form at least 60% of the breast contacting surface. Preferably, the deformable portions of the plurality of deformable portions form from 70% to 80% of the breast contacting surface.

In the example shown in FIG. 2 the deformable portion 216 is formed from a thermoplastic elastomer of Shore A hardness 30. The thickness of the deformable portion 216 is 1.25 mm.

In the example shown in FIG. 2, the deformable portions 216 form 75% of the breast contacting surface 210.

The opening 220 leads to a conduit 230 extending in a direction away from the breast contacting surface 210. The conduit 230 is adapted to accommodate a nipple received in the opening 220 for expressing milk.

The breast contacting element 200 includes a distal opening 238 for operably connecting the breast contacting element 200 to a fluid pressure source. The distal opening 238 provides an outlet to the conduit 230. The distal opening 238 is fluidly connected to a negative pressure source so as to convey fluid pressure from the negative pressure source to the opening 220. Optionally, the distal opening may fluidly connect the opening to a container so to collect milk expressed from breast during use.

Each channel 222 extends towards the distal opening 238 from the opening 220. This arrangement provides a further advantage that the channel 222 extends sufficiently from the opening 220 such that a distal end of the channel 222 is beyond the user's nipple. Optionally, and as shown in the example of FIG. 2 (b), a conduit 230 extends from the opening to the distal opening 238.

The breast contacting element 200 is arranged so that breast contacting surface 210 flares outwardly from the opening 220. The breast contacting surface 210 extends to a perimeter edge 218. Thus, the perimeter edge 218 is radially and axially spaced from the opening 220. In this way, there is provided a concave breast contacting surface 210 extending around the opening 220 which more comfortably conforms to the user's breast. By conforming with the breast, the breast can easily seal against the breast contacting surface 210 when engaged therewith.

The breast contacting element 200 includes a first flared surface 281 extending proximal to the opening 220. The first flared surface extends outward at a first angle relative to the central longitudinal axis A-A of the breast contacting element. In the example, the first angle is 15°.

The breast contacting element 200 includes a second flared surface 282 extending proximal to the opening 220. The second flared surface extends outward at a second angle relative to the central longitudinal axis A-A of the breast contacting element. In the example, the second angle is 60°. The channel 222 extends from the opening to the second flared surface. The channel 222 thereby extends from the first flared surface and beyond a transition surface between the first flared surface and the second flared surface. In this way, the open channel is advantageously oriented so that, with a breast engaged against the breast contacting surface 210, at least a portion of the channel remains unblocked. Fluid pressure from the opening 220 is thus conveyed to each deformable portion 216 throughout use.

In alternative arrangements, the breast contacting element, or a part thereof, may be configured to provide any suitably shaped breast contacting surface to engage the breast in a comfortable manner and/or to create an effective seal between the breast and breast contacting element. In this way, the breast contacting surface may be a recessed cone, dome, hemisphere or any hybrid shape thereof. The concave surface of the breast receiving surface may be symmetrically-shaped or may be asymmetrically-shaped, for example as described below in reference to FIG. 11.

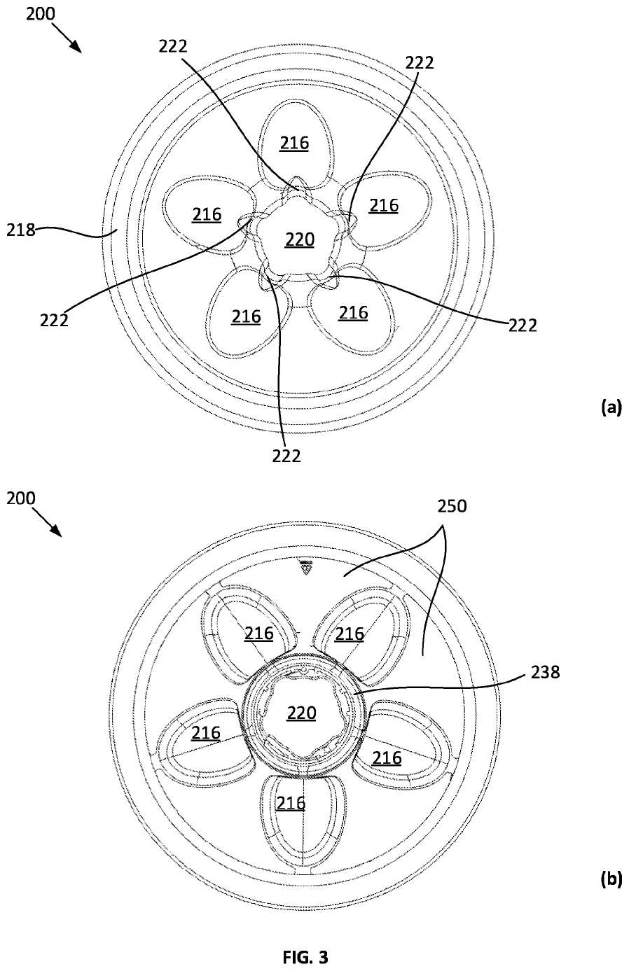

Referring now to FIGS. 3 (a) and 3 (b), there are shown top and bottom views of the breast contacting element 200 of FIG. 2. The deformable portions 216 are arranged equidistantly around the opening 220, separated by regions of a support element 250.

The channels 222 are also arranged equidistantly around the opening so as to provide a channel 222 for each deformable portion 216. Each channel 222 extends from a respective deformable portion 216, through the opening 220 to the distal opening 238. Furthermore, in the example shown, each channel 222 is recessed at the distal opening 238 so as to be able to fluidly connect each channel 222 to a corresponding recessed channel in a subsequent conduit or outlet fitted to the distal opening 238.

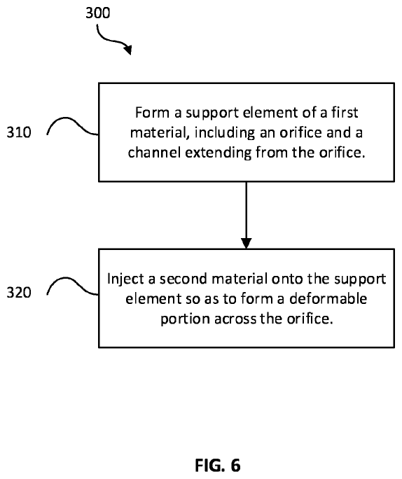

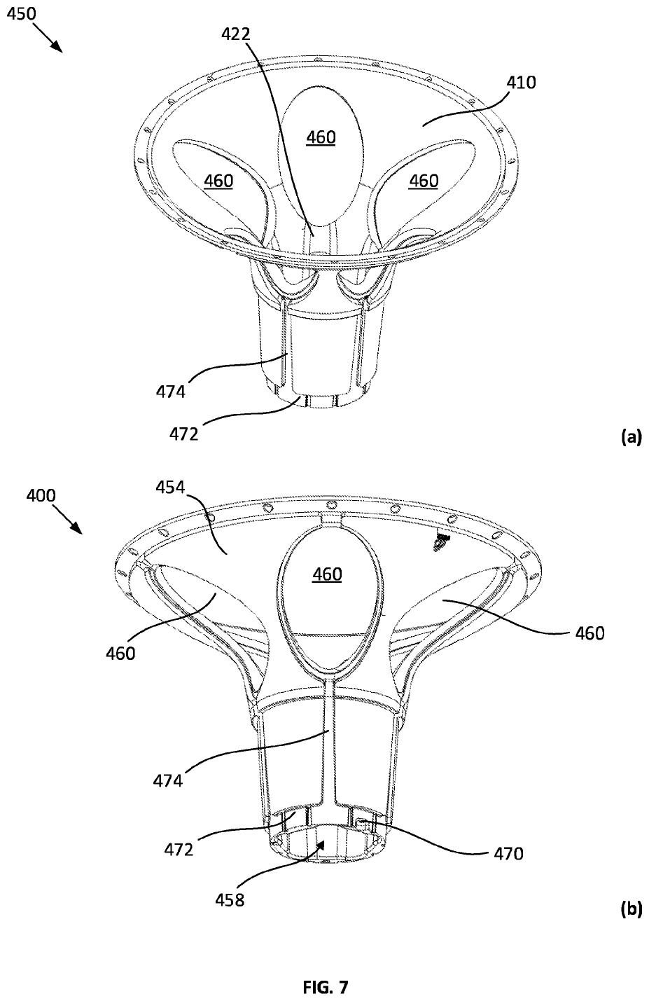

The support element 250 includes a plurality of orifices, as is described in further detail below in reference to FIGS. 6 and 7. Each deformable portion 216 is formed across a respective orifice such that the deformable portion 216 on the breast contacting surface of the breast contacting element 200 extends to the opposing, or underside, surface of the breast contacting element 200. In this way, when a fluid pressure is conveyed to the deformable portion 216, the opposing surface remains exposed to ambient pressure allowing a pressure difference to form across opposing surfaces of the deformable portion 216. The pressure difference thus initiates the flex response to the fluid pressure.

The breast contacting element 200 may include a textured region. In particular the textured region may be provided on the deformable portion 216 of the breast contacting surface 210 and/or the adjoining regions of the support element 250. In this way, the interaction of the breast contacting element 200 with the user's breast may be modified to provide increased comfort or reduced friction therebetween. The textured region may also enhance the massaging effect of the deformable portion, for example by use of projections, ribs or a raised pattern.

The breast contacting element 200 extends from the opening to a perimeter edge 218. The perimeter edge 218, in this particular case an annular perimeter edge, is formed of a deformable material so as to provide a cushioned surface for the user, and to improve expressing efficiency by enhancing the seal between the breast contacting element 200 and the user's breast. In the example shown, the perimeter edge 218 is formed from the same material as the deformable portion 216. In other words, the deformable portion 216 and perimeter edge are formed from a common deformable material so as to simplify the manufacturing process of the breast contacting element 200.

Referring now to FIG. 4 there is shown a further cross-sectional view of the breast contacting element 200 of FIG. 2 engaged with a breast 299 for expressing milk. Accordingly, the breast 299 is engaged with the breast contact element 210, including the deformable portion 216, such that the nipple 298 is received in the opening 220. The deformable portion 216 is domed inwardly, that is the deformable portion 216 is convex with respect to the breast contacting surface, thereby projecting towards the breast 299 in use.

The breast 299 is engaged in a first position without a fluid pressure provided from the opening 220. That is, the breast 299 is engaged with the breast contacting surface 210, ready for the negative pressure source to be activated. The channel 222 is not blocked by the breast.

The breast 299 extends towards the opening 220 to an innermost, or centremost, engagement limit 219 of the breast contacting surface 210 with the breast 299.

Referring now to FIGS. 5 (a) to 5 (d) there is shown a series of views of the arrangement of FIG. 4 showing a computer simulation of the response of the deformable portion 216 to a fluid pressure in the opening 220. The series of views show the response of the deformable portion 216 over a portion of a cycle of fluid pressure, in this case the negative pressure provided by the negative pressure source, as is commonly used with known breast pump assemblies.

The simulation was performed using a finite element analysis of a breast contact element in which the support element 250 was a polypropylene material and the deformable portions 21 were a thermoplastic elastomer material of Shore-A hardness of 30. The simulation further included providing an operational fluid pressure of 460 millibar to the opening 220. The breast 299 was simulated using a Mooney-Rivlin model in which the Mooney-Rivlin coefficients C01 and C10 for adipose tissue of the breast were defined as 1333 Pa and 2000 Pa respectively, as reported in Liu et al, Radiation Physics and Chemistry, 2017, 140, pp. 295-299.

A first view, FIG. 5 (a), corresponds to the first position of the breast in the breast contacting element 200 as described with reference to FIG. 4 above. The deformable portion 216 is engaged with the breast 299 prior to the application of a fluid pressure from the opening 220.

A second view, FIG. 5 (b), shows the arrangement of FIG. 4 at an initial time interval after a fluid pressure is provided to the opening 220. The fluid pressure has been conveyed to the deformable portion 216 by the channel 222 such that the deformable portion 216 responds to the fluid pressure by deflecting towards the breast 299. In particular, the channel 222 enables the deformable portion 216 to deflect even though the breast contacting element 200 is in contacting engagement with the breast because the fluid pressure is conveyed past the engagement limit 219 of the breast contacting element 200 with the breast. The deflection of the deformable portion 216 initiates a massaging pressure against the breast 299.

A third view, FIG. 5 (c), shows the arrangement at a subsequent time interval to the second view. The fluid pressure has increased from the fluid pressure at the initial time interval causing the deformable portion 216 to deflect further towards the breast 299 such that the massaging pressure against the breast 299 is increased. The increased massaging pressure pushes the nipple 298, and thereby the engagement limit 219, towards the distal opening (not shown). Nevertheless, the channel 222 remains unblocked by the breast 299 and continues to convey fluid pressure to the deformable portion 216. Furthermore, massaging pressure combined with a movement of the nipple 298 towards the distal opening provides a stripping action against the breast 299 which improves milk expression.

A fourth view, FIG. 5 (d) shows the arrangement at a further time internal to the third view. The fluid pressure has increased from the fluid pressure at the third view to a maximal fluid pressure. The maximal fluid pressure provides the deformable portion 216 with a maximal deflection towards the breast. The deformable portion 216 thereby provides a maximal massaging pressure against the breast.

Furthermore, the maximal massaging pressure pushes the nipple 298 to a maximal distance towards the distal opening, enhancing the stripping action. The movement of the nipple 298 moves the engagement limit 219 to a maximal distance towards the distal opening. Despite this, the channel 222 remains unblocked and continues to convey fluid pressure to the deformable portion 216.

To complete the fluid pressure cycle, after reaching a maximal fluid pressure the fluid pressure is released and returns to the initial pressure. Consequently, the deflection of the deformable portion 216 returns to its initial position and massaging pressure is released. The breast 299 reverts to its initial portion.

By repeated fluid pressure cycles the breast contacting element 200 is able to provide repeated or pulsed massaging effect to an increased area of the breast thereby enabling improved milk expression. The breast contacting element 200 may provide more efficient milk expression shortening expressing time for the user. Additionally, or alternatively, milk may be expressed using lower fluid pressures due to the increased massaging effect, resulting in the corresponding breast pump assembly being more comfortable for the user to use.

As will be understood, when providing a plurality of deformable portions 216 on the breast contacting element 200 each deformable portion 216 responds concurrently in the manner described above, thereby further enabling improved milk expression as well as increased efficiency and improved comfort for the user.

Referring now to FIG. 6, there is shown flow chart illustrating a method 300 of forming a breast contacting element for a breast pump assembly. The method 300 may be carried out to provide any of the breast contacting elements described herein.

A first step 310 of the method 300 includes forming a support element of a first material. The support element if formed such that it includes an orifice and a channel extending from the orifice.

A second step 320 of the method 300 includes injecting a second material onto the support element so as to form a deformable portion across the orifice. In this way, the breast contacting element is provided as a single part with deformable portions formed thereon. Furthermore, by providing channels on the support element which extend to the orifice, the breast contacting element is formed with channels extending from an opening to the deformable portion. In this way, a significant proportion of the deformable portion is exposed to a fluid pressure in use. Consequently, the deformable portion provides a reliable response to negative pressure conveyed thereto by the channel, even where the deformable portion is engaged with the breast.

In certain examples, the breast contacting element comprises a breast contacting surface and the deformable portion forms at least a portion of the breast contacting surface.

In certain examples, the orifice is a plurality of orifices and the first step 310 of forming the support element includes forming an inlet manifold for receiving the injected second material. Optionally, the inlet manifold is configured such that during the step of injecting the second material onto the support element the inlet manifold directs the injected second material to each of the plurality of orifices. In this way, the manufacturing is simplified because one inlet manifold directs the injected second material to multiple orifices.

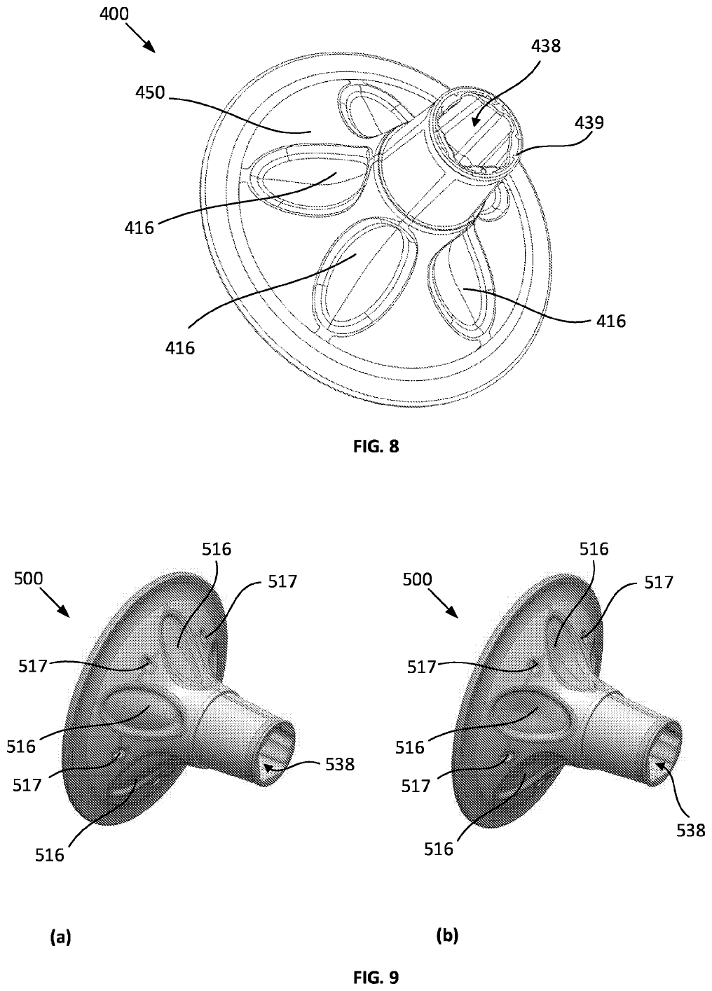

Furthermore, the inlet manifold may be configured such that during the step of injecting the second material onto the support element the inlet manifold directs the injected second material to any desired region of the support element. In this way, the second material may be used to form one or more aspects of the breast contacting element in a single manufacturing step. For example, the inlet manifold may be configured to direct the injected second material to form one or a plurality of deformable portions, a deformable flange or a sealing element, such as the sealing element described below in reference to FIG. 8.

In certain examples the support element provided by the method 300 is rigid. Preferably, the support element may be formed of a plastic, so as to minimise the weight of the breast contacting element. In this way, the support element provides dimensional stability to the breast contacting element. Consequently, one or more deformable portions may be provided on a breast contacting element so as to reliably provide improved massaging effect to desired areas of the breast and without requiring additional parts.

Optionally, the support element may be transparent so that the user can observe the expressed milk during use. In the example shown in FIG. 2, the breast contacting element 200 is formed of a transparent polypropylene material.

Referring now to FIGS. 7 (a) and (b), there is shown a support element 450 such as a support element formed in the first step 310 of the method 300 described in reference to FIG. 6. The support element 450 may be formed as a first step in manufacturing a breast contacting element. Certain aspects of the breast contacting element may be formed on the support element 450 in one or more subsequent manufacturing steps, such as the second step 320 of the method described in reference to FIG. 6.

The support element 450 includes an orifice 460 and a channel extending 422 from the orifice 460. The orifice 460 extends through the support element 450. That is, the orifice 460 extends from a breast contacting surface 410 of the support element 450 to the opposing, or underside, surface 454 of the support element 450.

The support element 450 includes an inlet manifold 470. The inlet manifold 470 is adapted to receive a second material in a second manufacturing step, for example the second step 320 shown in FIG. 6 of injection moulding the second material onto the support element 450.

The inlet manifold 470 is configured such that during the second manufacturing step the inlet manifold 470 directs the injected second material to specific regions of the support element 450. In this way, the second material may be directed to form one or more aspects of the breast contacting element, such as one or a plurality of deformable portions, a deformable flange or a sealing element.

The inlet manifold 470 is provided at a distal orifice 458 of the support element 450. However, in alternative arrangements, an inlet manifold 470 may be provided at any suitable position on the support element 450 such that a second material can be conveniently directed to form particular aspects of a breast contacting element.

In this example, the inlet manifold 470 includes provided within a recess 472 at the distal orifice 458. The recess 472 adjoins a series of recessed channels 474, each leading from the recess 472 to a respective orifice 460. In this way, the inlet manifold 470 is able to conveniently direct injected second material to one or more orifices 460.

Referring now to FIG. 8, there is shown a breast contacting element 400 formed from the support element 450 described with reference to FIGS. 7 (a) and (b). The second material is injected onto the support element 450 such that the second material forms a deformable portion 416 across an orifice 460 of the breast contacting element 400. In this way, the second material is provided in the second step 320 of the method described in reference to FIG. 6.

The second material is injected onto the support element 450 at the inlet manifold 470. The inlet manifold 470 is configured to direct the second material around the recess 472 at the distal orifice 458, so as to form a sealing element 439 around a distal opening 438 of the breast contacting element. In this way, the distal opening 438 is provided with a sealing element 439 to enable the distal opening 438 to be sealingly coupled to other aspects of a breast pump assembly. By using the second material to form the sealing element 439, the breast contacting element 400 has a simple design which requires no additional sealing components and is more convenient for the user.

In the example shown, the deformable portion 416 is coupled to an edge of the orifice 460. That is the second material forms a sheet provided across the orifice 460, such that the deformable portion 416 is self-supporting. The deformable portion 416 is thereby configured to move relative to the breast in response to a fluid pressure, such as the deflection described with reference to FIGS. 5 (a) to (d) above.

In alternative arrangements, the breast contacting surface may include only the second material, such that a single deformable portion extends circumferentially around the breast contacting surface. Optionally, the second material may extend over regions of a support element so as to provide a continuous surface of the second material on the breast contacting surface.

Referring now to FIGS. 9 (a) and (b), there is shown a further example breast contacting element 500. Where the features are the same as a previous example, the reference numbers are the same, other than the initial digit is a “5”. The breast contacting element 500 is substantially the same as the example shown in FIG. 2 with the exception that a plurality of protrusions 517 are provided on the breast contacting surface.

Each protrusion 517 is arranged to project from the breast contacting surface towards a user's breast when engaged with the breast contacting element. In this way, the protrusions apply a secondary massaging effect to further improve breast milk expression.

In the example shown the protrusions 517 are arranged to be interspersed between deformable portions 516, although any suitable number and arrangement of individual protrusions may be provided. Optionally, the protrusions may be formed from the second material so as to provide protrusions in a deformable material, thereby improving comfort for the user. It will be noted that the protrusions 517 may be optionally provided in combination with any deformable portion described herein.

Furthermore, and as shown in FIG. 9 (a), a deformable portion 516 of the breast contacting element 500 is domed outward. That is, the deformable portion 516 is recessed from, or concave to, the breast contacting surface in an initial position. The deformable portion 516 shown is independent to the protrusions 517 such that the deformable portion 516 may be provided on a breast contacting surface without any protrusions 517.

Upon application of a negative fluid pressure, the deformable portion 516 deflects towards the breast in response such that the deformable portion 516 inverts to be convex to the breast contacting surface, as shown in FIG. 9 (b). In this way, the deformable portion 516 is configured to change from a first position in which the deformable portion 516 is recessed from the breast, to a second position in which the deformable portion 516 projects towards the breast. Consequently, the deformable portion 516 provides a pronounced massaging effect on the breast.

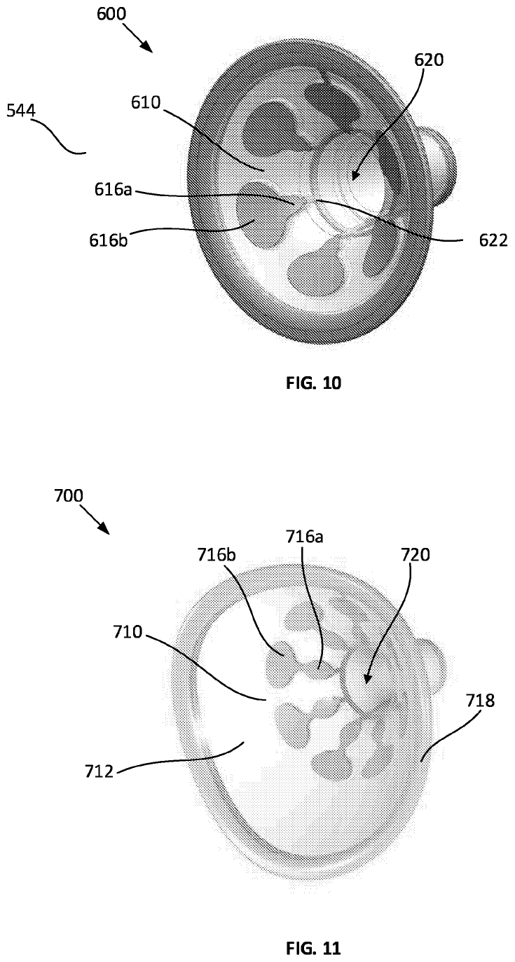

Referring now to FIG. 10, there is shown a further example breast contacting element 600. Where the features are the same as a previous example, the reference numbers are the same, other than the initial digit is a “6”. The breast contacting element 600 is substantially the same as the example shown in FIG. 2 with the exception that a deformable portion includes a first and second lobe 616a, 616b.

The first lobe 616a extends from the channel 622. That is, the first lobe 616a is provided proximal the opening 620. The first lobe may form a portion of the channel 622.

The second lobe 616b extends from the first lobe 616a. The second lobe 616b is positioned and configured to deflect in response to a fluid pressure so as to provide a massaging effect to the breast in the manner of other example deformable portions described herein.

Suitably, the first lobe 616a may also be configured to deflect in response to a fluid pressure. The first lobe 616a may be configured to deflect in a reduced manner to the second lobe 616b so as to provide different massaging effects to differing regions of the breast.

Referring now to FIG. 11, there is shown a further example breast contacting element 700. Where the features are the same as a previous example, the reference numbers are the same, other than the initial digit is a “7”. The breast contacting element 700 is substantially the same as the example shown in FIG. 10 other than that breast contacting element 700 further includes a flange portion 712. The breast contacting element 700 is asymmetrically shaped.

The flange portion 712 extends partially around the breast contact surface 710. That is, the flange portion 712 extends from the breast contact surface 710 so as to be adapted to partially enclose a lower region of the breast in use, thereby providing improved support to the breast.

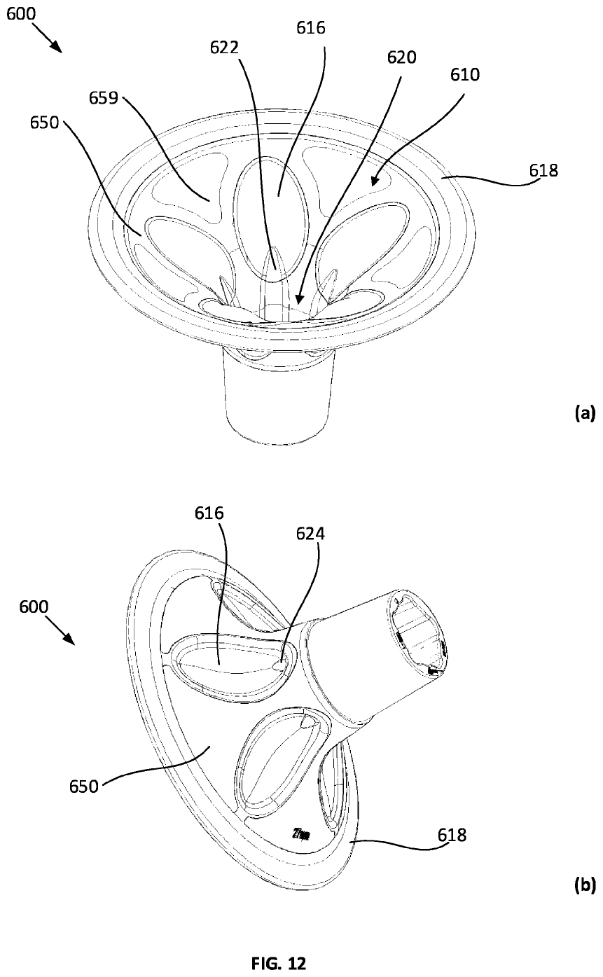

Referring now to FIGS. 12 (a) and 12 (b), there is shown a further example breast contacting element 600. Where the features are the same as a previous example, the reference numbers are the same, other than the initial digit is a “6”. The breast contacting element 600 is substantially the same as the example shown in FIG. 2 with the exception that the breast contact surface includes a plurality of textured regions.

The breast contacting element includes a plurality of deformable portions 616 on a breast contacting surface 610. Each deformable portion 616 of the plurality of deformable portions 616 is provided with a channel 622 configured to convey fluid pressure from the opening 620. In this example, the deformable portions 616 are arranged so as to be spaced apart around the breast contacting surface 610. In this way, the breast contacting surface 610 includes a plurality of deformable portions 616 separated by regions of a support element 650.

The breast contacting element 600 is arranged so that breast contacting surface 610 flares outwardly from the opening 620. The breast contacting surface 610 extends to a perimeter edge 618. Thus, the perimeter edge 618 is radially and axially spaced from the opening 620.

The breast contacting element 600 includes a plurality of textured surfaces. In particular textured surfaces are provided on each of the deformable portion 616 and the adjoining regions 659 of the support element 650. In this way, the interaction of the breast contacting element 600 with the user's breast may be modified to provide increased comfort or reduced friction therebetween.

The breast contacting element 600 extends from the opening to a perimeter edge 618. The perimeter edge 618, in this particular case an annular perimeter edge, is formed of a deformable material so as to provide a cushioned surface for the user, and to improve expressing efficiency by enhancing the seal between the breast contacting element 600 and the user's breast. The perimeter edge 618 is also provided with a textured surface extending substantially therearound.

The texture of each textured surface of the breast contacting element 600 is a random textured surface. In the example shown, each textured surface is provided using a texture rating of 30 BDI in the corresponding injection moulding tool.

It will be understood that textured surfaces are not limited or restricted to those specific regions of the breast contacting element described with respect to the example of FIG. 12. Any region or regions within the breast contacting surface may be provided with a suitable textured surface so as to modify the interaction of a respective region with a user's breast. Regions within a breast contacting surface may be provided with the same textured surface. Alternatively, regions may be provided with different textured surfaces so as to differently modify the respective interaction with the user's breast as is appropriate.

Each channel 622 of the breast contacting element 600 is formed in the breast contacting surface 610. In this way, the channel 622 forms an elongated cavity in the breast contacting surface 610. The channel 622 is provided as a recess extending along the breast contacting surface 610. That is, the channel 622 is formed as an open channel.

The channel 622 includes a profile shape such that, with a breast engaged against the breast contacting surface 610, at least a portion of the profile shape remains unblocked along the channel.

The breast contacting element 600 includes first and second flared surfaces in substantially the same manner as described above with reference to FIG. 2. Accordingly, the first flared surface extends outward at a first angle relative to the central longitudinal axis A-A of the breast contacting element and the second flared surface extends outward at a second angle. In the example, the first angle is 15°. In the example, the second angle is 60°.

Each channel 622 extends from the opening 620 to the second flared surface. Each channel 622 extends partially into the deformable portion 616. The channel 622 thereby extends from the first flared surface and beyond a transition surface between the first flared surface and the second flared surface.

Referring in particular to FIG. 12 (b), each deformable portion 616 includes a reinforcing portion 624. The reinforcing portion 624 is provided as an area of the deformable portion 616. The reinforcing portion 624 is aligned with the channel 622 and projects from the external surface of the deformable portion 616. A thickened wall is thereby provided around the interface of the channel 622 and the deformable portion 616 such that the deformable portion 616 is more reliably moulded. Manufacturing reliability is thereby increased.

It will be appreciated by persons skilled in the art that the above detailed examples have been described by way of example only and not in any limitative sense, and that various alterations and modifications are possible without departing from the scope of the invention as defined by the appended claims. Various modifications to the detailed examples described above are possible.

As will be apparent to the skilled person, the examples described herein may include any suitable number of deformable portions such that any breast contacting element may include one or a plurality of deformable portions. Furthermore, any feature ascribed to a single deformable portion may apply without limitation to some or all of a plurality of deformable portions of a breast contacting element.

A deformable portion may be provided in any suitable shape or orientation on the breast contacting surface. Non-limiting example shapes include oval, tear-drop, wedge-like, circular or any segment or sector of a circle.

Where a plurality of deformable portions is provided, non-limiting arrangements of the deformable portions on the breast contacting surface include providing deformable portions arranged circumferentially or radially around the opening, or arranged at varying circumferential or radial distances around the opening.

The opening may be fluidly connected to any suitable container so as to collect milk expressed from the breast during use, for example a bottle or a pouch.

It will be clear to a person skilled in the art that features described in relation to any of the examples described above can be applicable interchangeably between the different examples. The examples described above are to illustrate various features of the invention.

1. A breast contacting element for a breast pump assembly, comprising:
an opening adapted to receive a nipple of a user's breast;
a breast contacting surface extending from the opening and comprising a deformable portion for engaging the user's breast; and
a channel configured for conveying a fluid pressure from the opening to the deformable portion such that the deformable portion deflects in response to the fluid pressure applied thereto.

2. A breast contacting element according to claim 1, wherein the deformable portion is domed.

3. A breast contacting element according to claim 1, wherein the deformable portion is configured to invert or flex in response to the fluid pressure applied to the deformable portion.

4. A breast contacting element according to claim 1, wherein deformable portion has a thickness in the range of from 0.5 mm to 2 mm, preferably in the range of from 1 mm to 1.5 mm.

5. A breast contacting element according to claim 1, wherein the deformable portion comprises an elastomer having a material hardness in the range of from Shore A 20 to Shore A 80, and preferably in the range of from Shore A 25 to Shore A 50.

6. A breast contacting element according to claim 1, wherein the breast contacting surface comprises a plurality of deformable portions.

7. A breast contacting element according to claim 6, wherein the plurality of deformable portions comprise at least 60% of the breast contacting surface, and preferably comprise from 70% to 80% of the breast contacting surface.

8. A breast contacting element according to claim 1, the breast contacting element further comprising a support element comprising an orifice, and wherein the deformable portion is formed across the orifice.

9. A breast contacting element according to claim 8, wherein the orifice extends though the support element.

10. A breast contacting element according to claim 8, wherein the deformable portion is coupled to an edge of the orifice.

11. A breast contacting element according to claim 1, wherein the channel is formed in the breast contacting surface.

12. A breast contacting element according to claim 1, wherein the channel comprises a recess extending along the breast contacting surface.

13. A breast contacting element according to claim 1, wherein the breast contacting element comprises a distal opening for operably connecting the breast contacting element to a fluid pressure source, and wherein the channel extends towards the distal opening.

14. A breast contacting element according claim 13, wherein the breast contacting element comprises a conduit extending from the opening to the distal opening.

15. A breast contacting element according to claim 14, wherein the channel extends along an inner surface of the conduit.

16. A breast contacting element according to claim 1, wherein the breast contacting surface comprises a textured region.

17. A breast contacting element according to claim 16, wherein the textured region is provided on the deformable portion.

18. A breast pump assembly comprising:
a conduit, operably connectable to a fluid pressure source, the conduit having an opening adapted to receive a nipple of a user's breast,
a breast contacting surface extending from the opening and comprising a deformable portion for engaging the user's breast; and
a channel configured for conveying a fluid pressure generated by the fluid pressure source from the opening to the deformable portion such that the deformable portion deflects in response to the fluid pressure applied thereto.

19. A method of forming a breast contacting element for a breast pump assembly, the method comprising:
forming a support element of a first material, the support element comprising an orifice and a channel extending from the orifice,
injecting a second material onto the support element so as to form a deformable portion across the orifice.

20. A method according to claim 19, wherein the breast contacting element comprises a breast contacting surface, and wherein the deformable portion forms at least a portion of the breast contacting surface.

21. A method according to claim 19, wherein the orifice is a plurality of orifices and wherein the step of forming the support element comprises forming an inlet manifold for receiving the injected second material.

22. A method according to claim 21, wherein the inlet manifold is configured such that during the step of injecting the second material onto the support element the inlet manifold directs the injected second material to each of the plurality of orifices.

23. A method according to claim 21, wherein the breast contacting element comprises a distal opening for coupling the breast contacting element to a breast pump, and wherein the inlet manifold is configured to further direct the injected second material to form a sealing element at the distal opening.
